(19) United States
(12) Patent Application Publication
(10) Pub. No.: US 2019/0350887 A1
(43) Pub. Date: Nov. 21, 2019

(54) INCREASING BIOAVAILABILITY OF IRON WITH AVENANTHRAMIDE 2c

(71) Applicant: The Quaker Oats Company, Chicago, IL (US)

(72) Inventors: YiFang CHU, Glenview, IL (US); Lisa FLEIGE, Chicago, IL (US)

(21) Appl. No.: 15/983,589

(22) Filed: May 18, 2018

(51) Int. Cl. A61K 31/196; A61K 33/26; A61K 9/00; A23L 7/10; A23L 33/16

(52) U.S. Cl. CPC A61K 31/196; A61K 33/26; A23V 2002/00; A23L 7/198; A23L 33/16; A61K 9/0056

(57) ABSTRACT

Providing an orally deliverable composition that includes iron and avenanthramide 2c increases the bioavailability of the iron in the composition. Avenanthramide 2c can be delivered in an effective amount by selecting ingredients high in avenanthramide 2c or increasing the bioaccessibility of avenanthramide 2c in certain ingredients before including them in the composition.

### BACKGROUND

### 1. Technical Field

The present disclosure relates generally to the use of avenanthramide 2c to increase iron bioavailability in orally deliverable compositions, such as food and beverage products.

### 2. Description of Related Art

The balance of iron in the human body is driven by the amount absorbed from food and other enterally administered substances, because there is no mechanism for excretion of iron from the body. In turn, the amount of iron that the human body can absorb from enterally administered substances is driven by the bioavailability of the iron present in the substance. Iron bioavailability of a particular substance depends on a number of factors. Some compounds commonly found in food products have been found to be promoters of iron absorption, thereby increasing the bioavailability of iron, while other compounds inhibit iron absorption, which decreases iron bioavailability.

Iron bioavailability has been estimated to be in the range of 14-18% for mixed diets and 5-12% for vegetarian diets. See Hurrell R, Egli I. “Iron Bioavailability and Dietary Reference Values.” Am J Clin Nutr. 2010; 91(suppl): 1461S-7S. Hurrell and Egli also reported that polyphenols have been found to inhibit iron absorption in foods. Id. at 1462S. The same conclusion was reported by the World Health Organization, who state that “where phytate or polyphenol intakes are high, the risk of iron and zinc deficiencies increases because the bioavailability of both of these minerals from foods is reduced in the presence of these compounds.” World Health Organization. Guidelines on food fortification with micronutrients. Geneva, Switzerland: World Health Organization, 2006. at 141. Accord, Brune et al. Fe Absorption and Phenolic Compounds. 1989; Eur. J. Clin Nutr. 43:547-558.

Oats (Avena sativa) are a source of polyphenolic compounds called avenanthramides. Conventional knowledge in the art would suggest that the polyphenols in oats would inhibit iron absorption, thereby reducing the bioavailability of iron found in food products that contain oats.

### SUMMARY

The present invention relates to the surprising discovery that one of the three primary types of avenanthramides found in oats—avenanthramide 2c—is a promoter of iron bioavailability in compositions, and bioabsorption of iron in humans.

In an exemplary embodiment of the invention, a method for increasing bioabsorption of iron in a human comprises administering to the human a source of avenanthramide 2c comprising an effective amount of avenanthramide 2c to increase bioabsorption of iron found in an accompanying orally administered composition. In another exemplary embodiment, the source of avenanthramide 2c is oats. In still another exemplary embodiment, the oats comprise a total avenanthramide concentration, wherein the avenanthramide 2c comprises greater than 37% of the total avenanthramide concentration. In another exemplary embodiment, the source of avenanthramide 2c comprises synthesized avenanthramide 2c. In another exemplary embodiment, the source of avenanthramide 2c is a partially hydrolyzed oat flour that retains its whole grain status.

In one exemplary embodiment of the invention, a composition comprises a source of iron and a source of avenanthramide 2c, wherein avenanthramide 2c is present in the composition in an effective amount to increase bioavailability of the iron in the composition. In another exemplary embodiment, the composition is fortified with at least a portion of the iron. In yet another exemplary embodiment, the composition comprises at least one ingredient with a native iron content. In still another exemplary embodiment, the source of avenanthramide 2c is oats. In another exemplary embodiment, the oats comprise a total avenanthramide concentration, wherein the avenanthramide 2c comprises greater than 37% of the total avenanthramide concentration. In another exemplary embodiment, the source of avenanthramide 2c comprises synthesized avenanthramide 2c. In another exemplary embodiment, the source of avenanthramide 2c is a partially hydrolyzed oat flour that retains its whole grain status. In another exemplary embodiment, the composition is in a dosage form selected from the group consisting of a pill, a tablet, a cachet, a capsule, a granule, a pellet, a bead, a powder, a troche, a lozenge and a gel. In another exemplary embodiment, the composition is a food product. In another exemplary embodiment, the food product is in a form selected from the group consisting of a bar, a cookie, an oatmeal, a cereal, a cracker, a chip, a granola cluster, and a cake. In another exemplary embodiment, the food product is in the form of a beverage.

Thus, numerous embodiments of compositions and methods that increase or enhance iron bioavailability in orally administered substances are disclosed.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood from the Detailed Description, here below, with reference to the following illustrations of exemplary non-limiting embodiments wherein:

FIG. 1 illustrates the effect of the three primary oat avenanthramides on iron bioavailability in humans based on an in vitro Caco-2 cell line test model.

FIG. 2 illustrates the ability of Caco-2 cell line models to predict iron bioavailability in humans.

The above figures are provided for the purpose of illustration and description only, and are not intended to define the limits of the disclosed invention. Use of the same reference number in multiple figures is intended to designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the particular embodiment. The extension of the figures with respect to number, position, relationship, and dimensions of the parts to form the preferred embodiment will be explained or will be within the skill of the art after the following teachings of the present invention have been read and understood.

### DETAILED DESCRIPTION

The present disclosure makes reference to one or more specific embodiments by way of illustration and example. It is understood, therefore, that the terminology, examples, drawings and embodiments are illustrative and are not intended to limit the scope of the disclosure.

Various avenanthramide nomenclature systems have been proposed. The Dimberg system describes avenanthramides using a numerical descriptor for the anthranilic acid component, and a letter descriptor for the accompanying phenylpropanoid (“c” for caffeic acid; “f” for ferulic acid; “p” for anthranilic acid p-coumaric acid). Using the Dimberg system, the three primary avenanthramides found in oats are referred to as avenanthramide 2c, avenanthramide 2p, and avenanthramide 2f. These same avenanthramides are referred to under the Collins system as avenanthramide C, avenanthramide A, and avenanthramide B. The IUPAC names for each of these avenanthramides are 2-[[(E)-3-(3,4-dihydroxyphenyl)prop-2-enoyl]amino]-5-hydroxybenzoic acid, 5-hydroxy-2-[[(E)-3-(4-hydroxyphenyl)prop-2-enoyl]amino]benzoic acid, and 5-hydroxy-2-[[(E)-3-(4-hydroxy-3-methoxyphenyl)prop-2-enoyl]amino]benzoic acid, respectively. Although any of these naming systems could be used interchangeably to clearly describe avenanthramide compounds, the Dimberg nomenclature will be used throughout the remainder of this disclosure, and in the originally submitted claims.

As described above in the background, it was generally understood in the art that polyphenols are known to inhibit the ability of the human body to absorb iron when polyphenols are consumed with the iron. The present invention involves the surprising discovery that one of the three main avenanthramides found in oats—avenanthramide 2c—in fact promotes the bioabsorption of iron in humans. The improvement effects of the invention are described herein in terms of improving the bioabsorption of iron in humans, or improving the bioavailability of iron in compositions, as appropriate. The terms “bioabsorption” and “bioavailability” can, depending on context, be used interchangeably.

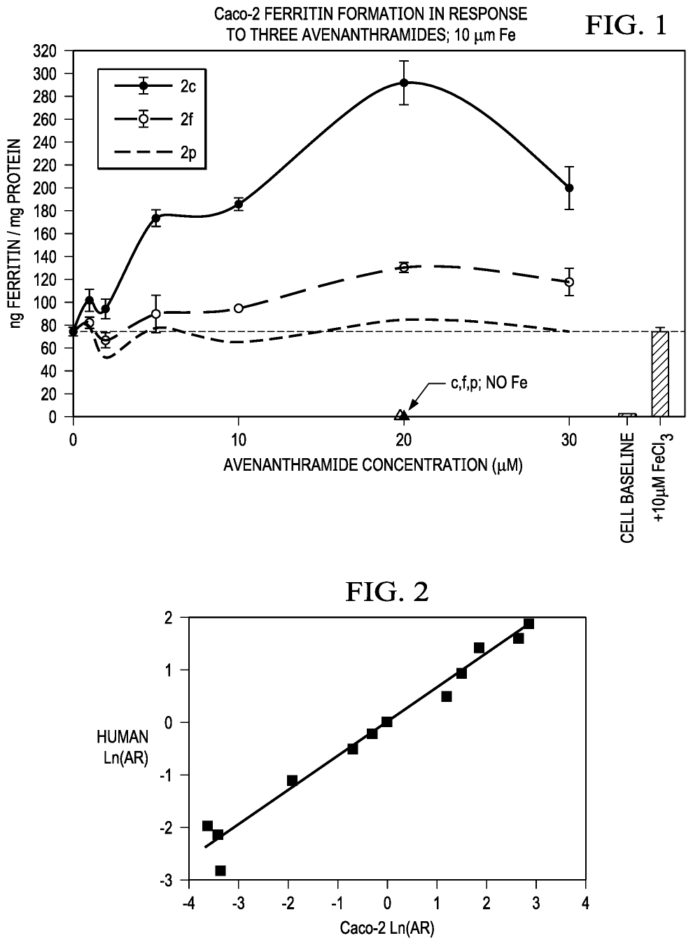

FIG. 1 is a graph illustrating the results of in vitro tests that were performed in order to predict the effect that avenanthramide 2c, avenanthramide 2p, and avenanthramide 2f would have on iron bioabsorption in humans. Specifically, an in vitro Caco-2 cell bioassay was used that accurately predicts how a compound will influence iron bioabsorption in humans. In these cells, the intracellular iron storage protein known as ferritin forms in direct proportion to cell iron uptake.

In the experimental protocol, a 10 μM ferric chloride solution is administered to Caco-2 bacterial cells. The ferric chloride solution is administered to the cells by itself (as a control) and along with the compound(s) being tested to assess their effect iron bioavailability, as measured by the amount of ferritin formed in the cells—a sensitive and accurate measurement of cell iron uptake. The test protocol measures the total weight of cell protein and the total weight of ferritin produced by the cells in order to generate a weight ratio of ferritin to cell protein (in units of ng ferritin formed per mg cell protein). The ratios from the test samples are compared against a control sample wherein only ferric chloride is administered to the cells. Test samples with higher ferritin values (ng ferritin/mg cell protein) compared to the control indicate improvement or enhancement of iron bioavailability, whereas test samples with ferritin values approximately equal to (or within the margin of error) or less than the control indicates no effect on iron bioavailability or inhibition of iron bioavailability, respectively.

As seen in FIG. 1, at very low concentration for all avenanthramides, the measured effect on iron bioavailability was very small to negligible. However, at a concentration of 5 μM, it is seen that avenanthramide 2c exhibited a large increase in iron bioavailability—more than double the control value. Further, when the concentration of avenanthramide 2c was increased to 20 μM, ferritin formation increased by a factor of more than 1.5 over the 5 μM sample (or, more than 3.5 times the control value). An increase of avenanthramide 2c concentration to 30 μM dropped the ferritin value back towards the ratio measured at 10 μM, but still above the values measured at 10 μM and 5 μM, and well above control.

This effect was particularly surprising in view of previous literature on the subject of how polyphenols affect iron bioavailability. Polyphenols are generally assumed by those skilled in the art to have no effect or to actually inhibit non-heme iron bioavailability. It was also surprising in view of the fact that the other two avenanthramides tested—2p and 2f—exhibited negligible or zero effect on iron bioavailability under the same testing conditions.

FIG. 2 is a graph that compares the iron absorption ratio measured using the Caco-2 cell bioassay described above with the iron absorption ratio measured in humans. The absorption ratio is defined as the absolute value of the test sample divided by the absolute value of the control sample. In particular, the graph of the natural log of the absorption ratio in Caco-2 versus the natural log of the absorption ratio in humans displays an excellent linear fit, with a slope of approximately 0.6401. In other words, an equation that allows conversion between absorption ratio in humans and absorption ratio in Caco-2 is as follows: ln(absorption ratio in humans)=0.6401×ln(absorption ratio in Caco-2). See Yun S, Habicht J P, Miller D D, Glahn R P. An in vitro digestion/Caco-2 cell culture system accurately predicts the effects of ascorbic acid and polyphenolic compounds on iron bioavailability in humans. J Nutr 2004; 134:2717-21.

The surprising findings of the Caco-2 testing described above form part of the basis for the embodiments of the invention described herein. In a general sense, the findings indicate that including a source of avenanthramide 2c along with an iron source in an orally administered or deliverable substance would increase the bioavailability of the iron, thereby improving the ability to balance iron levels in the human body using less total iron intake.

One way to realize the improvement in iron bioavailability is to provide an orally deliverable composition in the form of a food product or beverage that includes as an ingredient or component thereof, a source of avenanthramide 2c at an amount that is effective to increase iron bioavailability. In one embodiment, the source of avenanthramide 2c is an oat-based ingredient (such as oat flour) made from oats with naturally high levels of avenanthramide 2c.

The Caco-2 results shown in FIG. 1 indicate that one example of an effective amount of avenanthramide 2c in an orally deliverable composition can range, in one embodiment, from 2.8 mg of avenanthramide 2c per mg of iron to 16.8 mg of avenanthramide 2c per mg of iron. These amounts were calculated based on the molar ratios of avenanthramide 2c to iron in the Caco-2 models and the molecular weights of avenanthramide 2c and iron. The Caco-2 model also indicates that the maximal effect on iron bioavailability would be found, in one embodiment, at about 11.2 mg of avenanthramide 2c per mg of iron. These example effective amounts are not limiting on the present invention in its broadest sense because other amounts may be effective depending on the presence or absence of other iron bioavailability promoters or inhibitors in the orally deliverable composition.

The level of each specific avenanthramide present in oats varies widely according to many factors, including the variety of the oat and growing conditions. The avenanthramide contents of 109 samples of oats were measured to determine whether oat selection would be a viable option for increasing iron bioavailability using avenanthramide 2c. A summary of this testing is provided in the table below.

TABLE 1
Summary of Avenanthramide Measurements in Oats

| | Ave 2c | Ave 2p | Ave 2f | Total Ave |
|---|---|---|---|---|
| | (μg/g) | (μg/g) | (μg/g) | (μg/g) |
| Average Value | 12.28 | 9.08 | 11.38 | 32.75 |
| Median Value | 9.17 | 7.20 | 10.02 | 26.53 |
| Minimum Value | 0.72 | 0.73 | 0.92 | 2.88 |
| Maximum Value | 153.04 | 64.07 | 79.85 | 296.95 |

Table 1 shows a summary of the avenanthramide analysis of 109 samples of oats, which represented approximately 33 oat varieties grown in various locations throughout North American and the United Kingdom. As seen therein, avenanthramide concentrations can vary widely between different oat samples. Further, the percentage contribution of avenanthramide 2c to the total avenanthramide concentration was calculated. The average and median percentage contribution of avenanthramide 2c to the total avenanthramide concentration across the 109 samples was 37%. The minimum and maximum percentage contributions were 11% and 70%, respectively.

Thus, it can be said that any oat sample for which the percentage contribution of avenanthramide 2c to the total avenanthramide concentration is greater than 37% is high in avenanthramide 2c content. As such, in one embodiment of the present invention, a composition comprises an oat-based ingredient using oats that comprise a percentage contribution of avenanthramide 2c to total avenanthramide concentration which is greater than 37% in an amount effective to increase iron bioavailability in the composition.

In another embodiment, instead of or in addition to selecting oats with high levels of avenanthramide 2c for inclusion into a composition, oats can be modified to make the avenanthramide 2c already present in the oats more bioaccesible than it is when the oats are in native form. For example, it has been found that when whole grain oats are partially hydrolysed by enzymatic hydrolysis while retaining their original whole grain characteristics, the bioaccessibility of the avenanthramide 2c present in the oats increased by between about 2% and 15%.

The amount of bioaccessible avenanthramides in control and test oat flour samples were measured by reversed-phase high-performance liquid chromatography (HPLC). Processed oat flour was extracted, dried, and re-suspended in 80% ethanol. Aliquots were analyzed by HPLC using a chromatography column equipped with a diode array detector. The peaks corresponding to avenanthramides 2c, 2p, and 2f were quantified by comparing the obtained peak areas to those of standard curves.

An example of a process for preparing an oat flour comprising hydrolyzed starch that retains its whole grain status is found in U.S. patent application Ser. No. 12/264,399 filed Nov. 4, 2008 and issued as U.S. Pat. No. 8,574,644 on Nov. 5, 2013, the content of which is expressly incorporated herein by reference in its entirety, as an example.

Generally, in one embodiment, oat flour comprising hydrolyzed starch that retains its whole grain status can be made according to the following method: Whole oat flour is conditioned with water, steam and alpha-amylase enzyme in a continuous mixer, and heated to a temperature of about 165° F. The moisture content of the mixture can be between 28% and 32%. The conditioned mixture is then extruded in a commercial scale twin screw extruder, and the temperature of the mixture is further increased to about 285° F. to inactivate the enzyme. The extruded dough was then formed into pellets, dried to a moisture content of less than 10%, and ground into particles to produce a soluble oat flour that retains its whole grain status.

In some embodiments, the soluble whole oat flour maintains its standard of identity as whole grain throughout processing (e.g., starch hydrolysis, pelletizing, drying, and/or grinding). “Whole grain” or “standard of identity as whole grain” shall mean that the oat grain, “consists of the intact, ground cracked or flaked caryopsis, whose principal anatomical components—the starchy endosperm, germ and bran—are present in approximately the same relative proportions as they exist in the intact caryopsis.” (See, AACC International's Definition of “Whole Grains,” approved in 1999, available at http://www.aaccnet.org/initiatives/definitions/pages/wholegrain.aspx (last accessed Feb. 11, 2016).) Further, if the principal nutrients (i.e., starch, fat, protein, dietary fiber, beta-glucan, and sugar) are present in approximately the same relative proportions for a partially hydrolyzed oat and the original oat, it can be assumed that the processed oat maintains its whole grain status. However, since the average molecular weight of starch (e.g., amylopectin) in whole grains varies widely among whole grain oat products, a shift in starch moieties from higher molecular weight to lower molecular weight does not alter whole grain status if the total starch content remains the same. Therefore, in one embodiment of the present invention, a composition for increasing iron bioavailability includes an oat-based ingredient, such as oat flour, that is partially hydrolyzed but retains whole grain status.

In another embodiment, avenanthramide 2c may be produced in pure form (as the term “pure” is understood to include normal manufacturing tolerances as to impurities, etc.) using a number of different methods. For example, bacterial cells, such as E-coli cells, engineered specifically for the purpose can be used to produce avenanthramide 2c. Alternatively, synthetic organic chemistry methods using, for example, 3, 4-dihydroxycinnamic acid and 5-hydroxyanthranilic acid as precursor reactants can be used to produce avenanthramide 2c.

It can be understood from the present disclosure that the ingredient high in avenanthramide 2c may be combined with virtually any food or beverage product containing iron in order to increase the bioavailablity of that iron. The iron in the inventive composition may, in one embodiment, be native to the ingredients used therein. In another embodiment, the composition is fortified with iron, for example, using one or a combination of electrolytic iron, ferrous fumarate/sulfate, bisglycine chelate, or NaFeEDTA. In another embodiment, the composition contains both a native iron source and a fortified iron source.

The invention may be embodied in other specific forms without departing from the spirit or essential characteristics thereof. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the recitation of method steps does not denote a particular sequence for execution of the steps. Such method steps may therefore be performed in a sequence other than recited unless the particular claim expressly states otherwise. In view of the foregoing, through one or more various aspects, embodiments and/or specific features or sub-components, the present disclosure is thus intended to bring out one or more of the advantages that will be evident from the description.

### ADDITIONAL DESCRIPTION

The following paragraphs are offered as further description of the various embodiments of the disclosed invention.

In a first embodiment, a method for increasing bioabsorption of iron in a human comprises administering to the human a source of avenanthramide 2c comprising an effective amount of avenanthramide 2c to increase bioabsorption of iron found in an accompanying orally administered composition.

In a second embodiment according to any other embodiment, combination of embodiments, or subcombination of embodiments disclosed herein, the source of avenanthramide 2c is oats.

In a third embodiment according to any other embodiment, combination of embodiments, or subcombination of embodiments disclosed herein, the oats comprise a total avenanthramide concentration, wherein the avenanthramide 2c comprises greater than 37% of the total avenanthramide concentration.

In a fourth embodiment according to any other embodiment, combination of embodiments, or subcombination of embodiments disclosed herein, the source of avenanthramide 2c comprises synthesized avenanthramide 2c.

In a fifth embodiment according to any other embodiment, combination of embodiments, or subcombination of embodiments disclosed herein, the source of avenanthramide 2c is a partially hydrolyzed oat flour that retains its whole grain status.

In a sixth embodiment, a composition comprises a source of iron and a source of avenanthramide 2c, wherein avenanthramide 2c is present in the composition in an effective amount to increase bioavailability of the iron in the composition.

In a seventh embodiment according to any other embodiment, combination of embodiments, or subcombination of embodiments disclosed herein, the composition is fortified with at least a portion of the iron.

In an eighth embodiment according to any other embodiment, combination of embodiments, or subcombination of embodiments disclosed herein, the composition comprises at least one ingredient with a native iron content.

In a ninth embodiment according to any other embodiment, combination of embodiments, or subcombination of embodiments disclosed herein, the source of avenanthramide 2c is oats.

In a tenth embodiment according to any other embodiment, combination of embodiments, or subcombination of embodiments disclosed herein, the oats comprise a total avenanthramide concentration, wherein the avenanthramide 2c comprises greater than 37% of the total avenanthramide concentration.

In an eleventh embodiment according to any other embodiment, combination of embodiments, or subcombination of embodiments disclosed herein, the source of avenanthramide 2c comprises synthesized avenanthramide 2c.

In a twelfth embodiment according to any other embodiment, combination of embodiments, or subcombination of embodiments disclosed herein, the source of avenanthramide 2c is a partially hydrolyzed oat flour that retains its whole grain status.

In a thirteenth embodiment according to any other embodiment, combination of embodiments, or subcombination of embodiments disclosed herein, the composition is in a dosage form selected from the group consisting of a pill, a tablet, a cachet, a capsule, a granule, a pellet, a bead, a powder, a troche, a lozenge and a gel.

In a fourteenth embodiment according to any other embodiment, combination of embodiments, or subcombination of embodiments disclosed herein, the composition is a food product.

In a fifteenth embodiment according to any other embodiment, combination of embodiments, or subcombination of embodiments disclosed herein, the food product is in a form selected from the group consisting of a bar, a cookie, an oatmeal, a cereal, a cracker, a chip, a granola cluster, and a cake.

In a sixteenth embodiment according to any other embodiment, combination of embodiments, or subcombination of embodiments disclosed herein, the food product is in the form of a beverage.

1. A method for increasing bioabsorption of iron in a human, the method comprising administering to the human a source of avenanthramide 2c comprising an effective amount of avenanthramide 2c to increase bioabsorption of iron found in an accompanying orally administered composition.

2. The method of claim 1, wherein the source of avenanthramide 2c is oats.

3. The method of claim 2, wherein the oats comprise a total avenanthramide concentration, wherein the avenanthramide 2c comprises greater than 37% of the total avenanthramide concentration.

4. The method of claim 1, wherein the source of avenanthramide 2c comprises synthesized avenanthramide 2c.

5. The method of claim 1, wherein the source of avenanthramide 2c is a partially hydrolyzed oat flour that retains its whole grain status.

6. A composition comprising a source of iron and a source of avenanthramide 2c, wherein avenanthramide 2c is present in the composition in an effective amount to increase bioavailability of the iron in the composition.

7. The composition of claim 6 wherein the composition is fortified with at least a portion of the iron.

8. The composition of claim 6 wherein the composition comprises at least one ingredient with a native iron content.

9. The composition of claim 6 wherein the source of avenanthramide 2c is oats.

10. The composition of claim 9 wherein the oats comprise a total avenanthramide concentration, wherein the avenanthramide 2c comprises greater than 37% of the total avenanthramide concentration.

11. The composition of claim 6 wherein the source of avenanthramide 2c comprises synthesized avenanthramide 2c.

12. The composition of claim 6 wherein the source of avenanthramide 2c is a partially hydrolyzed oat flour that retains its whole grain status.

13. The composition of claim 6 wherein the composition is in a dosage form selected from the group consisting of a pill, a tablet, a cachet, a capsule, a granule, a pellet, a bead, a powder, a troche, a lozenge and a gel.

14. The composition of claim 6 wherein the composition is a food product.

15. The composition of claim 13 wherein the food product is in a form selected from the group consisting of a bar, a cookie, an oatmeal, a cereal, a cracker, a chip, a granola cluster, and a cake.

16. The composition of claim 13, wherein the food product is in the form of a beverage.
